(19) United States
(12) Patent Application Publication
(10) Pub. No.: US 2024/0197289 A1
(43) Pub. Date: Jun. 20, 2024

(54) PORTABLE MEDICAL IMAGING DEVICE, METHOD OF USE AND SYSTEM

(71) Applicant: CAPERAY MEDICAL (PTY) LTD, Cape Town (ZA)

(72) Inventor: Christopher Leonard VAUGHAN, Cape Town (ZA)

(21) Appl. No.: 18/554,981

(22) PCT Filed: Apr. 8, 2022
(86) PCT No.: PCT/IB2022/053310

§ 371 Date: Oct. 11, 2023

Related U.S. Application Data
(60) Provisional application No. 63/173,825, filed on Apr. 12, 2021.

(51) Int. Cl. A61B 8/00; A61B 8/08; G16H 50/20

(52) U.S. Cl. CPC A61B 8/4427; A61B 8/403; A61B 8/4461; A61B 8/466; A61B 8/5207; A61B 8/5223; A61B 8/5269; G16H 50/20

(57) ABSTRACT

A portable imaging device and method for imaging biological tissue is provided. The device includes a scanning element having an ultrasound transducer configured to capture image data of biological tissue of a subject. The device includes a scanning surface serving as an interface between the scanning element and the biological tissue. A drive system is configured to automatically move the scanning element along the scanning surface. The scanning element is configured to capture image data as it is moved along the scanning surface by the drive system. The device includes an electronics module in data communication with the scanning element and with a computing device interface. The electronics module is configured to receive image data of the scanning element and transmit image data to a computing device via the computing device interface. The computing device operatively receives the image data and presents it to an operator in a processed form.

### CROSS-REFERENCE TO RELATED APPLICATIONS

This application claims priority from U.S. provisional patent application No. 63/173,825 filed on 12 Apr. 2021, which is incorporated by reference herein.

### FIELD OF THE DISCLOSURE

The invention disclosed herein relates to medical imaging. More specifically, it relates to medical imaging devices and methods used in the medical imaging of breast tissue. It may find exemplary application in its use in the detection of breast cancer.

### BACKGROUND

Medical screening examinations are performed on a patient to detect whether any signs of disease, such as breast cancer, are present. Screenings are usually done during routine check-ups and the signs of a disease, if any, are preferably identified before the patient shows any symptoms. The main aim of a screening test is to identify the disease at an early stage when the disease is still treatable and may potentially be cured.

As screenings have become more commonly used in the early detection of cancer, especially as an initial check for breast cancer, and with relatively promising accuracy, there is strong evidence to support the screening of women for breast cancer.

It is well-established that a key factor for successful treatment of breast cancer is early detection. Evidence thereof can be found in the results of screening programs that have been operating in Europe and North America for the past four decades (see for example Kopans DB, “Breast cancer screening: Where have we been and where are we going? A personal perspective based on history, data and experience” Clinical Imaging, 2018). Breast screening programs have not been offered to women in developing countries. Accordingly, such women have suffered, and continue to suffer, a high burden of the disease because their cancer is often detected too late.

The success of a breast-screening program is influenced by at least 6 factors: (1) high sensitivity and specificity (approaching 100% true positive and 100% true negative results); (2) high throughput (e.g. 5 women per hour); (3) operator independence; (4) being readily available in the community; (5) low cost to acquire and operate; and (6) rapid availability of results.

A favoured imaging modality is full-field digital mammography (FFDM) which comprises an X-ray of the breast which is, in some cases, followed up with a hand-held ultrasound (HHUS). FFDM suffers from poor sensitivity for women with dense breast tissue and is not widely available in the developing world because of its high acquisition cost. HHUS is highly dependent on the skill of the operator and is time consuming as it requires about 20 to 30 minutes to make a diagnosis. These drawbacks clearly undermine the factors affecting a successful program enumerated above.

As mentioned above, mammography is an imaging technique that uses planar X-rays, to detect early signs of breast cancer. X-ray mammography has been relatively successful in detecting breast cancer with the percentage of true positives being up to 90% and with the percentage of true negatives being up to 95%. However, mammography is known to perform at much lower accuracy when the patient's breasts have a high tissue density, which is often the case for younger women (i.e., women under the age of 50). The sensitivity of mammography is therefore known to be as low as 50%, the percentage of false negatives returned, due to tumours being obscured by overlapping tissues.

An imaging modality that has the ability to distinguish between tissues of different densities is ultrasound, commonly referred to as sonography. Although ultrasound lacks the spatial resolution of X-rays, it has been used as an adjunct to X-ray mammography for an extended period of time in practice.

Due to the ability of ultrasound to distinguish between tissues of different densities, diagnostic breast ultrasound tests play a crucial role in the detection and treatment of breast cancer in both young and older women. Traditionally, breast ultrasound tests have been conducted using a handheld probe which is manually operated and moved by an operator across the patient's breast. This approach, however, suffers from repeatability problems and the test results are influenced by the skill of the operator, as mentioned above.

In order to mitigate the possibility of operator errors, and other shortcomings of HHUS, automated breast ultrasound (ABUS) systems have been developed and implemented. It is well-known that such automated systems have gone some way in assisting clinicians with diagnostic decisions. However, these ABUS systems have at least two very important drawbacks associated therewith in that they are bulky in size and relatively expensive. These drawbacks limit their application in clinical care, particularly so in developing countries and rural areas.

Accordingly, the applicant considers there to be scope for improvement.

The preceding discussion of the background to the invention is intended only to facilitate an understanding of the present invention. It should be appreciated that the discussion is not an acknowledgment or admission that any of the material referred to was part of the common general knowledge in the art as at the priority date of this application.

### SUMMARY

In accordance with this disclosure there is provided a portable imaging device for imaging biological tissue, comprising:
a scanning element including an ultrasound transducer configured to capture image data of biological tissue of a subject;
a scanning surface configured to provide a surface serving as an interface between the scanning element and the biological tissue;
a drive system configured to automatically move the scanning element along the scanning surface, wherein the scanning element is configured to capture image data as the scanning element is moved along the scanning surface by the drive system; and
an electronics module in data communication with the scanning element and with a computing device interface, the electronics module being configured to receive image data of the scanning element and to transmit image data to a computing device via the computing device interface,
wherein the computing device operatively receives the image data and presents it to an operator in a processed form.

In one embodiment, the computing device is an integral computing device in data communication with the electronics module via the computing device interface. In another embodiment the portable imaging device is configured to receive a mobile computing device, removably securable thereto, and configured for the computing device interface to be connected to the mobile computing device.

The computing device may include a processor for processing the data to be presented to the operator in the processed form. The data in the processed form may include three-dimensional ultrasound images of the biological tissue which may be generated by performing a reconstruction algorithm on the image data captured by the scanning element.

The scanning surface may include a substantially planar mesh membrane configured to, in use, be pressed against a surface of the biological tissue so as to compress the tissue and enable the capturing of image data of a larger area of biological tissue.

In use the biological tissue and/or mesh membrane may be coated with a coupling agent, such as ultrasound gel or lotion. The mesh membrane may be manufactured from a mesh material providing sufficient structural integrity so that the scanning surface may compress the biological tissue during use, as required.

The imaging device may further include an optically translucent view panel, configured to provide an operator of the device with an unobstructed view of the surface of the biological tissue, and thereby enable the operator to position the device at a desired position with reference to the biological tissue to be scanned. The imaging device may alternatively include a video capturing device in data communication with the computing device which enables real-time display of the biological tissue on a display of the computing device, and thereby enables the operator to position the device at a desired position with reference to the biological tissue to be scanned.

The electronics module may include an analog front end and a digital beamformer. The analog front end may be configured to receive the image data as an output from the scanning element and filter, amplify and/or convert the image data from an analog to digital format. The digital beamformer may be an ultrasound beamformer configured to transmit and receive ultrasound waves of the scanning element for improved image data quality.

The imaging device may include a switch mechanism for controlling image data capture of the scanning element. The imaging device may include a pair of handles enabling the operator to manually carry/move the imaging device. Further features provide for the switch mechanism to be incorporated into the handles.

The imaging device may further include a controller arranged to control the drive system so as to move the scanning element continuously through a range of pre-selected positions to capture the image data, wherein the image data is two-dimensional ultrasound image data.

The scanning surface may have dimensions substantially equal to the dimensions of the largest biological tissue to be measured when the subject lies in a supine position.

The computing device may include a communications interface for operation of the computing device in a networked environment so as to enable transfer of data between the computing device and one or both of the imaging device and a server computer.

The communications interface may be configured to transmit at least one of the image data and/or the image data in processed form to a storage component. In one embodiment, the storage component is a cloud network storage maintained by the server computer. In another embodiment, the storage component is an on-board storage device, such as a memory of the computing device.

The computing device may be configured to interact with a backend medical service provided at the server computer and integrated with a medical service via the communications interface. The medical service may cooperate with a patient database and provide the operator of the imaging device with additional information associated with the subject via the computing device.

A further feature provides for the imaging device to be battery powered; and for the battery to be a rechargeable battery. In some embodiments, the battery may be housed in one of the handles.

In accordance with a further aspect of the invention there is provided a kit comprising:
a portable imaging device including:
   a scanning element including an ultrasound transducer configured to capture image data of biological tissue of a subject;
   a scanning surface configured to provide a surface serving as an interface between the scanning element and the biological tissue;
   a drive system configured to automatically move the scanning element along the scanning surface, wherein the scanning element is configured to capture image data as the scanning element is moved along the scanning surface by the drive system; and
   an electronics module in data communication with the scanning element and a computing device interface, the electronics module being configured to receive image data of the scanning element and to transmit image data to a computing device via the computing device interface, wherein the portable imaging device is arranged to receive a computing device; and
a computing device configured to operatively receive the image data from the electronics module via the computing device interface and process the image data of the biological tissue into a form presentable to an operator of the imaging device.

The computing device may be a tablet computer or a smart phone.

In accordance with another aspect of the invention there is provided a method for imaging and evaluating biological tissue, the method being performed at a server computer and comprising the steps of:
receiving image data of biological tissue captured by a portable imaging device;
performing computer-assisted diagnosis (CAD) on the image data by executing either or both of machine learning algorithms and artificial intelligence algorithms on the image data, the algorithms being configured to identify one or more abnormalities in the image data;
making a diagnosis on the server computer;
determining that the diagnosis is made within an acceptable confidence factor and sending the diagnosis to the portable imaging device and optionally a human data reviewer, or
determining that the diagnosis is not made within an acceptable confidence factor and sending the diagnosis to a human data reviewer, receiving input from the reviewer to accept or correct the diagnosis, and sending the diagnosis to the imaging device; and
updating either or both of the machine learning algorithms and the artificial intelligence algorithms with data points from the diagnosis.

The step of receiving image data of biological tissue captured by the portable imaging device may include the server computer receiving an analysis instruction and accessing a storage component to retrieve the image data. The server computer may receive the analysis instruction from the imaging device. Alternatively, the server computer may receive the analysis instruction from an operator of the imaging device.

The step of making a CAD on the server computer may include returning a positive finding if one or more abnormalities are present in the image data; and returning a negative finding if no abnormalities are present in the image data.

In one embodiment the diagnosis is made within an acceptable confidence factor if a negative finding is returned; and the diagnosis is not made within an acceptable confidence factor if a positive finding is returned. In another embodiment the diagnosis is made within an acceptable confidence factor if the confidence factor is higher than a pre-determined threshold. Similarly, the diagnosis is not made within an acceptable confidence factor if the confidence factor is lower than the pre-determined threshold.

In accordance with another aspect of the invention there is provided a system for imaging biological tissue, including a server computer having a memory for storing computer-readable program code and a processor for executing the computer-readable program code, the server computer comprising:
a data receiving component for retrieving image data image data of biological tissue captured by a portable imaging device;
a CAD component for performing CAD on the image data by executing either or both of machine learning algorithms and artificial intelligence algorithms on the image data, the algorithms being configured to identify abnormalities in the image data;
a diagnosis making component for making a diagnosis on the CAD;
a confidence factor determining component for determining that the diagnosis is made within an acceptable confidence factor and sending the diagnosis to the portable imaging device and optionally a human data reviewer, or determining that the diagnosis is not made within an acceptable confidence factor and sending the diagnosis to a human data reviewer, receiving input from the reviewer to accept or correct the diagnosis, and sending the diagnosis to the imaging device; and
an updating component for updating either or both of the machine learning algorithms and the artificial intelligence algorithms with data points from the diagnosis.

The system may include an instruction receiving component for receiving an analysis instruction and accessing a storage component to retrieve the image data.

Exemplary embodiments will now be described, by way of example only, with reference to the accompanying drawings.

### BRIEF DESCRIPTION OF THE DRAWINGS

In the Drawings:

FIG. 1 is a schematic block diagram of an example embodiment illustrating components of an imaging device;

FIG. 2A is a top view of a first example embodiment of an imaging device;

FIG. 2B is a section view of the imaging device of FIG. 2A along line B-B;

FIG. 2C is a side view of the imaging device of FIG. 2A;

FIG. 2D is a section view of the imaging device of FIG. 2A along line C-C;

FIG. 2E is a perspective view of the imaging device of FIG. 2A in which a scanning surface has been removed to reveal internal features;

FIG. 2F is perspective view of the imaging device of FIG. 2A in which side walls of the device have been removed to reveal internal features;

FIG. 3 is an illustration of an operator preparing the imaging device of FIG. 2A for use on a patient;

FIG. 4 is an illustration of the operator in FIG. 3 using the imaging device on the patient;

FIG. 5A is a top view of a second example embodiment of an imaging device;

FIG. 5B is a section view of the imaging device of FIG. 5A along line E-E;

FIG. 5C is a side view of the imaging device of FIG. 5A;

FIG. 5D is a section view of the imaging device of FIG. 5A along line D-D;

FIG. 5E is perspective view of the imaging device of FIG. 5A in which side walls of the device have been removed to reveal internal features;

FIG. 5F is a perspective view of the imaging device of FIG. 5A in which a scanning surface has been removed to reveal internal features;

FIG. 6A is a perspective view of a disassembled scanning element that may be used with an imaging device;

FIG. 6B is a perspective view of an assembled scanning element of FIG. 6A;

FIG. 6C is a perspective view of the scanning element of FIG. 6A with a communication interface;

FIG. 7A is a perspective view showing a top and rear of a beamformer that may be used with the imaging device;

FIG. 7B is a perspective view showing a bottom front of the beamformer of FIG. 7A connected to a transducer connector;

FIG. 8 is a flow diagram of an example method of using the device in a screening operation;

FIG. 9 is a flow diagram of an example method of steps carried out by a computing device used by the imaging device;

FIG. 10 is a high-level component diagram of a computing device used by the imaging device;

FIG. 11 is a flow diagram of an example method of steps carried out by a server computer in a system for imaging and evaluating biological tissue;

FIG. 12 is a high-level component diagram of a server computer arranged to execute the method of FIG. 11;

FIG. 13 illustrates an example screening workflow for the detection of abnormalities in biological tissue;

FIG. 14 illustrates example image data showing an abnormality in biological tissue; and

FIG. 15 illustrates an example of a computing device in which various aspects of the disclosure may be implemented.

### DETAILED DESCRIPTION WITH REFERENCE TO THE DRAWINGS

The disclosure provides an automated portable imaging device for imaging biological tissue. More specifically, the disclosure describes a portable automated breast ultrasound (ABUS) device for the use in diagnosis of carcinoma or other abnormalities in breast tissue.

The imaging device may be portable in that it is light weight, compact and comprises a plurality of handles for carrying the device during operation. The imaging device may further be physically small so as to facilitate portability and storage of the device. For example, the device may be sized so as to fit into a standard sized carry case or briefcase.

The imaging device may include at least one scanning element, such as an ultrasound transducer, which is configured to transmit and receive ultrasound waves to capture image data of biological tissue. The scanning element may be in data communication with a drive system configured to automatically move the scanning element across a biological tissue surface. In some embodiments a plurality of scanning elements may be used to capture image data.

The drive system may be configured to receive instructions from a controller to enable continuous movement of the scanning element through a range of pre-selected positions to capture image data in the form of two-dimensional ultrasound images.

The device may further include a scanning surface which may be pressed against a surface of the biological tissue during use of the device. The scanning surface may be optically translucent so that placement of the device at a desired location of the biological tissue is possible. For example, in the case of a breast tissue scan, the device needs to be centred over the nipple of the subject/patient for optimal results.

As soon as the device has been located at the desired position, the operator may initiate the scanning process to capture the image data.

The captured image data may be sent to an electronics module, including an analog front end and a digital beamformer. The electronics module may be configured to amplify, filter and convert the analog image data into a digital format which is processed and dynamically transmitted from the beamformer to a computing device via a computing device interface. The computing device may be integral with the imaging device and in data communication with the electronics module via the computing device interface. In some embodiments, the imaging device may be configured to receive the computing device, which is removably securable thereto, and configured for the computing device interface to be connected to the computing device. The computing device may be configured to receive image data from the electronics module via the computing device interface and process the image data of the biological tissue. The computing device interface may be a standard connector, such as a USB connector. The computing device may be a tablet computer or a smart phone, for example.

The computing device may include a processor and memory configured to process the digital image data by performing a reconstruction algorithm on the digital image data so as to generate three-dimensional ultrasound images of the biological tissue. The three-dimensional ultrasound images may then be displayed to an operator. The ultrasound images may be stored, for example on an on-board storage device such as a physical memory of the computing device, or a cloud network storage, and be accessible to a remote server computer for diagnoses of the patient by a trained clinician. In some embodiments, an initial diagnosis of the patient may be made by means of machine learning algorithms being performed on the ultrasound images.

The terms subject and patient may be used interchangeably to refer to a person, for example a woman, being the subject of a breast tissue scanning operation.

Exemplary portable imaging devices and a method of using the devices are now described in a non-limiting manner with reference to the Figures.

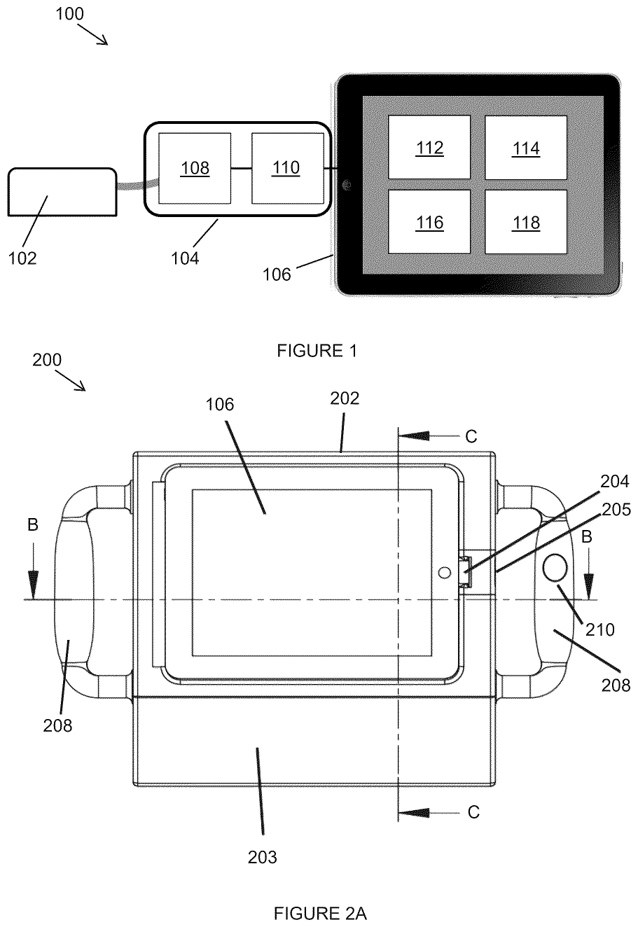

FIG. 1 shows a schematic block diagram of an example embodiment illustrating components of an imaging device (100). The imaging device (100) may include a scanning element (102), an electronics module (104), and a computing device (106).

In the present embodiment, the scanning element (102) includes an ultrasound probe, commonly referred to as a transducer, having a piezoelectric crystal acting as a transmitter and detector. As described in more detail below, the scanning element (102) is an automated ultrasound probe and configured to, in use, capture image data, in the form of two-dimensional images of biological tissue. Even though only one scanning element is shown, it should be appreciated that in some embodiments a plurality of scanning elements may be used to capture image data.

The scanning element (102) is in data communication with the electronics module (104). The scanning element (102) is in communication with the electronics module (104) by means of a data cable, such as a twisted pair cable, coaxial cable, fibre optic cable, or the like, which enables the transfer of power and data between the scanning element (102) and the electronics module (104). In some embodiments, the scanning element may be in wireless communication with the electronics module, such as via a Bluetooth or Wi-Fi™ wireless connection, or the like.

The electronics module (104) includes an analog front end (108) that serves as an interface between the scanning element (102) and other digital components of the imaging device (100). The analog front end (108) is configured to receive the image data, in the form of an analog signal, from the scanning element (102) and amplify, filter and convert the analog signals into a digital format, by means of an analog-to-digital converter, readable by the digital components. The image data is filtered by means of any suitable filter, such as an active low pass filter, band filter, adaptive filter or the like.

The electronics module (104) further includes a beamformer (110) configured to receive the converted digital signals and transmit the digital signals to the computing device (106) for processing. The beamformer (110) is a digital beamformer, such as an ultrasound beamformer, configured to focus and receive the converted signals of the scanning element (102) for improved image data quality.

The electronics module (104) is connected to the computing device (106) by means of the computing device interface. In the present embodiment the computing device interface is a standard connector pair, such as a USB cable and connector. It should be appreciated that in some embodiments, the computing device interface may be a wireless communication module, such as a Bluetooth module, allowing wireless communication between the electronics module and the computing device.

The converted output of the scanning element (102), in other words, the digital image data, is transmitted to the computing device (106) by means of the computing device interface. The computing device (106) may be any suitable computing device (such as a smartphone, tablet computer, or the like) capable of conducting data processing tasks. The computing device (106) provides a digital back end (112) capable of converting the received digital image data into readable tissue data by means of a set of instructions executed by a processor of the computing device (106). The set of instructions may be computer readable code having preconfigured algorithms and steps for converting the digital image data of the scanning element (102) into the readable tissue data. This may, for example, include performing a reconstruction algorithm on the image data captured by the scanning element so as to generate three-dimensional ultrasound images of the biological tissue. The processed data is then presented to the operator of the device in its processed form. In some embodiments, this may include the data being displayed to the user as three-dimensional ultrasound image data or as a data report in a format readable to the operator. In some embodiments, the processed data may be presented to the user as three-dimensional ultrasound images and as a data report.

It should be appreciated that the computing device may, in some embodiments, be configured to interact with a backend medical service provided at a medical server and integrated with a medical service. The medical service may cooperate with a patient database and provide an operator of the device with additional information associated with the subject undergoing the scanning operation.

In some embodiments, the computing device (106) may include a communications interface for operation of the computing device (106) in a networked environment so as to enable transfer of data between the components of the imaging device (100) and, for example, a server computer. Data transferred via the communications interface may be in the form of signals, which may be electronic, electromagnetic, optical, radio, or other types of signal.

The communications interface may be configured for connection to wireless communication channels (e.g., a Bluetooth network, wireless local area network (e.g., using Wi-Fi™), etc.) and may include an associated wireless transfer element, such as an antenna and associated circuitry.

It should be appreciated that the computing device (106) may be configured to communicate with the components of the device, or the server, through Wi-Fi, Bluetooth, Ethernet, a serial port and a variety of other interfaces to ultimately connect devices.

The computing device (106) is configured to display the ultrasound images, and the above-mentioned additional information if any, to an operator of the device (100) via a display (114) of the computing device (106) showing the underlying tissues being scanned by the scanning element.

In some embodiments, the computing device (106) may be configured to transmit the image data to a storage component via the communications interface. The storage component may be an on-board storage component, or it may be a remote storage component, such as a cloud storage network, or a database, which is maintained at a server computer, for example, the medical server.

The computing device (106) includes a controller (116) configured to send control instructions to components of the device, such as the scanning element (102) and/or a drive system. The controller (116) may, for example, receive instructions from an operator of the imaging device (100) to initiate a scanning operation and transmit instructions to the drive system to move the scanning element (102) from one side to another side of the device (100).

In some embodiments, the computing device (106) may include a user interface (118) which enables the operator to interact with the computing device and thereby control operation of the device.

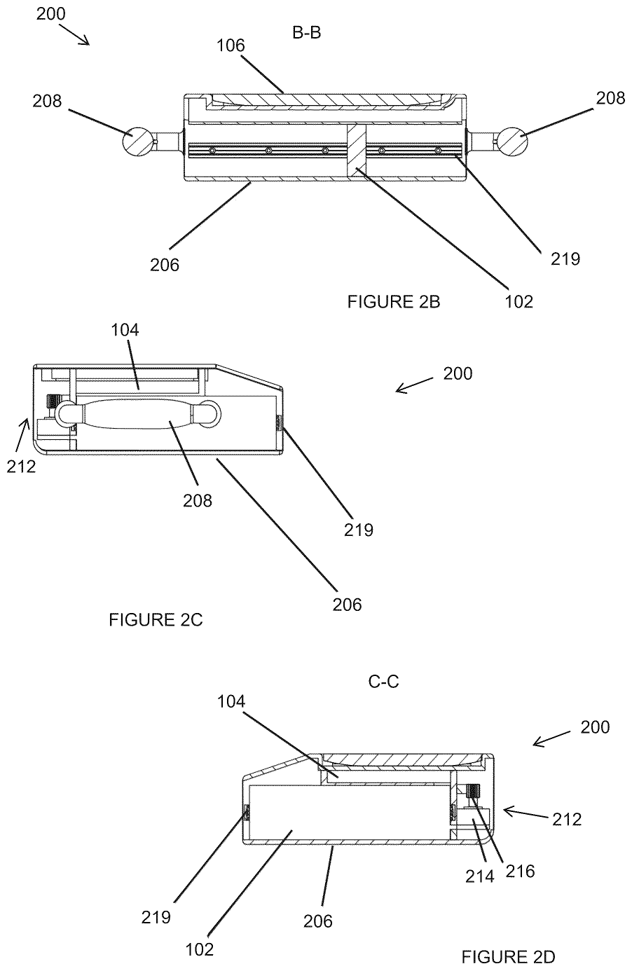

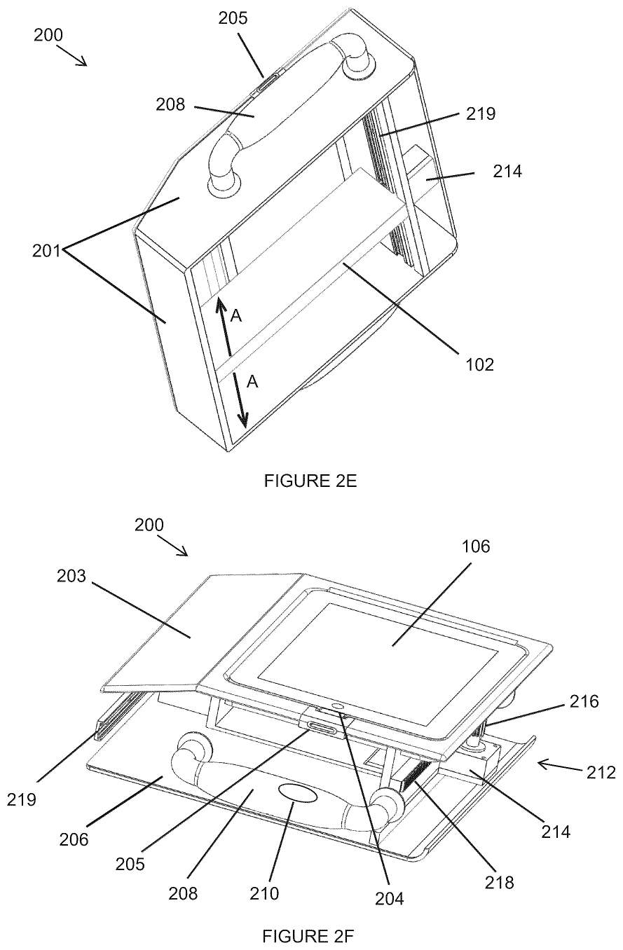

FIGS. 2A to 2F show a first example embodiment of an imaging device (200) as described herein.

The imaging device (200) includes an enclosure (202) for housing at least some of the components and protecting the components from damage. The enclosure (202) includes a body with side walls (201), a viewing panel (203), located at a top of the imaging device and a scanning surface (206). The scanning surface (206) includes a mesh membrane having a substantially planar surface and is configured to be received by the imaging device (200). In use, the mesh membrane is pressed against a surface of the biological tissue, such as the breast surface of a patient. In practice, the biological tissue and/or mesh membrane may be coated with a coupling agent, such as ultrasound gel or lotion to improve image capturing, as explained below. The mesh membrane is removably attachable to the imaging device. This enables an operator, or any other person, to remove the mesh membrane for sterilisation or disposal thereof, whichever the case may be. In some embodiments the mesh membrane may be permanently secured to the imaging device and sterilised by means of a sterilisation cloth, or the like.

In order to ensure that the operator may correctly locate the imaging device (200) over the biological tissue, the scanning surface (206) may be manufactured from an optically transparent material. The term optically transparent should be widely interpreted to mean both semi-transparent and fully transparent.

Typically, the nipple of the breast may be used as a marker for correct placement of the device. It may therefore be preferable for the mesh membrane to be manufactured from an at least partially optically translucent mesh material, such as nylon, polyester, or the like. This may enable the nipple to be visible through the mesh, particularly once coated with a coupling agent such as ultrasound gel, and enable the operator to properly position the imaging device by locating the nipple. In some embodiments, the meshed material may be coated with a strengthening material, such as polyvinyl chloride, to increase the durability of the meshed material.

The viewing panel (203) may be adequately located, at a top of the device (200), and also manufactured from an optically transparent material to enable an operator to see through the enclosure (202) and locate the device at a desired position.

The remainder of the enclosure (202) may be made from any suitable rigid material, such as high-density polyethylene, polypropylene, or the like. Ideally the imaging device (200) should be a compact and light-weight device to increase the portability of the device and, accordingly, the enclosure (202) should preferably be manufactured from a lightweight material.

The computing device (106) is housed within the enclosure (202) and connected to other components, such as the electronics module (104), of the imaging device (200) by means of a computing device interface (204) and to other external components, such as a server computer, via an external communication interface (205). It should be appreciated that the computing device interface and the external communication interface may be any suitable interface enabling data transfer as described above. The interfaces may be capable of transferring data in any direction and should not necessarily be limited to unidirectional transferring of data. In some embodiments, in which the interface includes a wired connector, the connector may be configured to electrically power at least some of the components of the device (200). In an embodiment in which the interface includes a wireless module, the imaging device (200) may include an on-board battery (not shown) for powering some of the components of the device, as will be described in more detail below. The electronics module (104) may also be housed within the enclosure (202).

The imaging device (200) further includes a pair of handles (208) to aid portability of the device and allow an operator to carry the device during scanning operations. In some embodiments the on-board battery, mentioned above, may be located within the handles (208). An advantage hereof is that by locating the battery in one of the handles (208) the bulkiness of the device (200) may be reduced. The battery may be a rechargeable battery having a connection point for directly connecting the battery to a source of power to charge the battery. In some embodiments the battery may be easily removeable and could therefore be recharged when removed from the device, or replaced in the case of a non-chargeable battery.

The scanning element (102) is orientated parallel to the pair of handles (208) and moveable from one side of the imaging device (200) to the other side, as discussed below. The scanning element (102) is located adjacent the scanning surface (206) (and in close proximity therewith to enable optimal scanning capabilities). In order to improve the acoustic coupling between the scanning element (102) and the biological tissue, an appropriate material such as ultrasound coupling gel may be added between the scanning surface (206) and the biological tissue.

The scanning element (102) is in data communication with the computing device (106), including a controller (116), for controlling image data capturing of the scanning element. In some embodiments image data capture instructions may be transmitted to the controller (116) of the computing device (106) by pressing a switch mechanism, such as a button (210), preferably located on at least one of the handles (208). For example, once the device has been located in a preferred position, the operator may press the button (210) which transmits a capture signal to the controller which in turn transmits instructions to the scanning element (102) and associated components thereof.

The scanning element (102) is further connected to a drive system (212) configured to enable continuous linear movement of the scanning element in a pre-configured direction. The drive system (212) may for example be in communication with the controller (116) of the computing device (106) which enables the drive system (212) to allow for linear movement of the scanning element (102) through a range of pre-configured positions. The drive system (212) may for example include a motor (214), such as a stepper motor, servo motor, brushless direct current motor, or the like, which rotates a pinion gear (216) configured to engage with a rack gear (218) to thereby allow linear movement of the scanning element. In order to ensure smooth and controlled movement of the scanning element (102), the imaging device (200) is fitted with a set of guide rails (219) configured to guide the scanning element (102) across the scanning surface (206). It should be appreciated that even though a rack and pinion gear combination is shown, any other suitable gear arrangement may be implemented which enables linear movement of the scanning element.

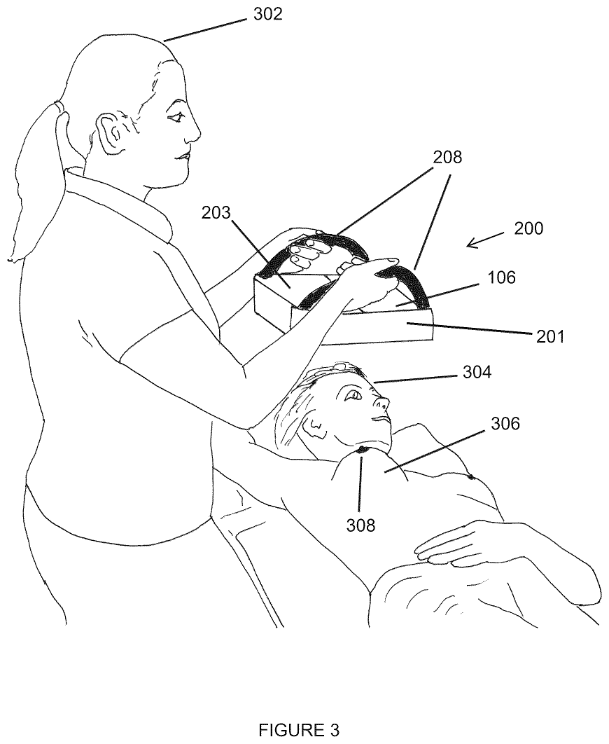

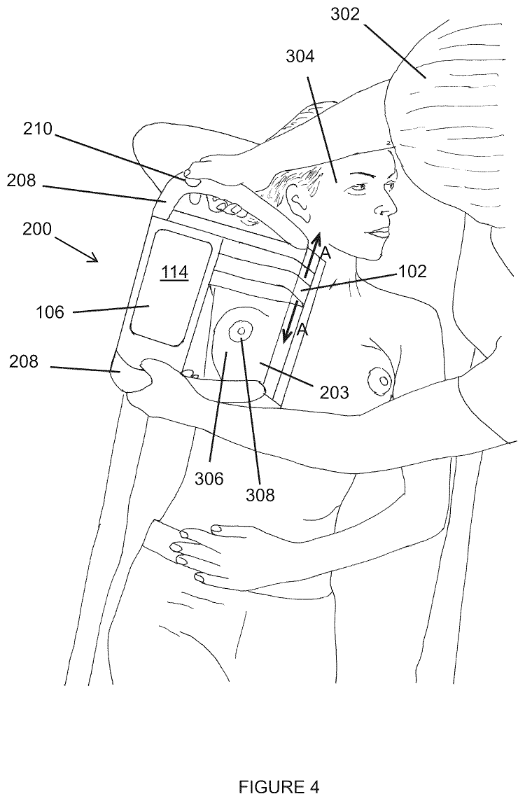

FIGS. 3 and 4 show illustrations of the first example embodiment of the portable imaging device (200) in use. The device (200) includes a pair of handles (208) which enable an operator (302) to position the device above the subject/patient (304) to be scanned. The computing device (106) is included at an upper surface of the device (200) adjacent to the optically translucent viewing panel (203). As shown in FIG. 4, the viewing panel (203) enables the operator to see the biological tissue (306), in this case the subject's breast, and locate the device (200) centrally over the subject's nipple (308) for optimal results.

After the operator (302) has located the device (200) in the desired position, the operator may initiate image data capture by pressing the button (210) located in at least one of the handles (208). As described above, the pressing of the button initiates the scanning progress and the scanning element (102) may then be guided from side-to-side, at right angles to the handles, as indicated by the arrows (A). Once the image data has been captured, it is processed by the computing device (106) and displayed on a display (114) thereof.

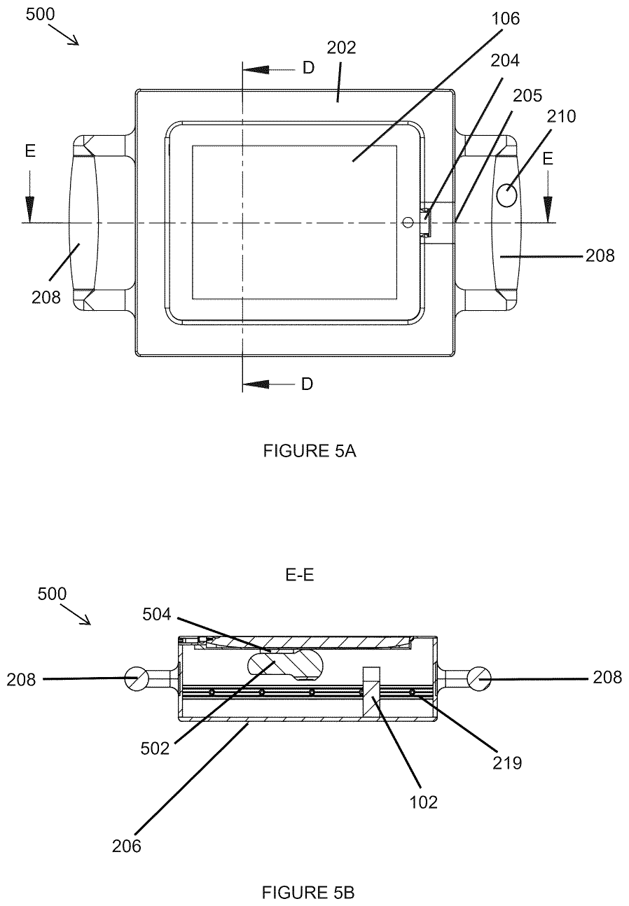

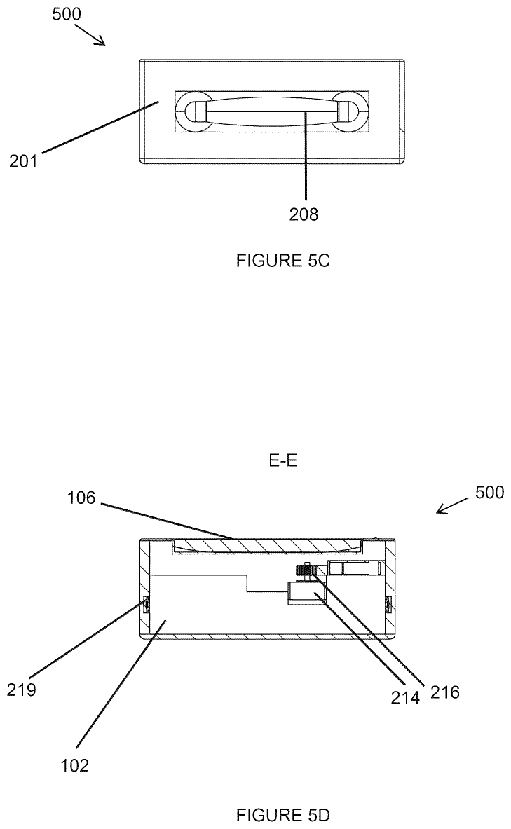

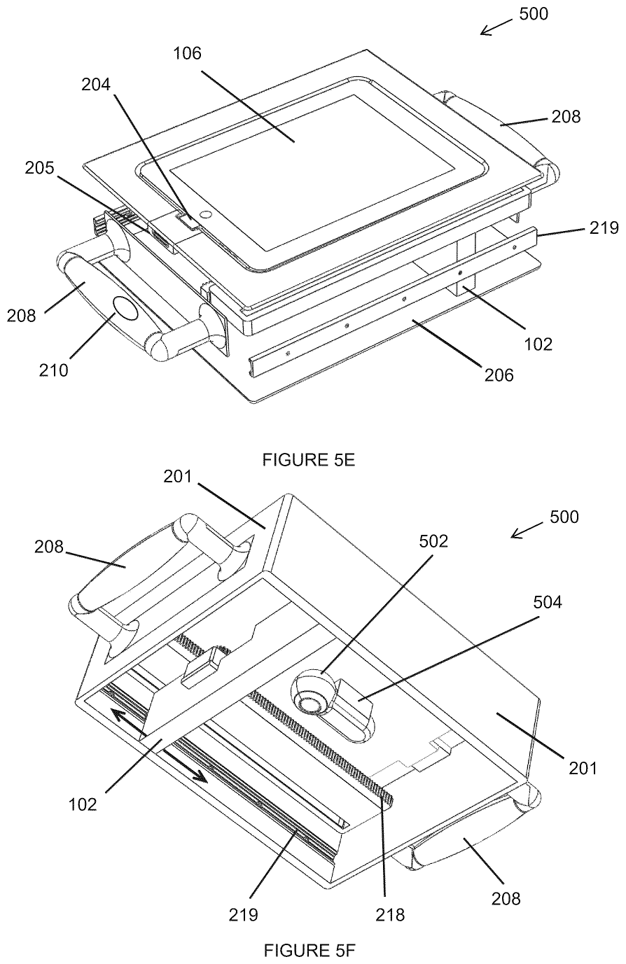

FIGS. 5A to 5F show a second example embodiment of a imaging device (500) as described herein.

The imaging device (500) as described with reference to FIGS. 5A to 5F may be substantially similar to the imaging device (200) described with reference to FIGS. 2A to 2F, and like features are indicated by like reference numerals.

The imaging device (500) includes a video capturing device (502), preferably a wide-angle video camera, instead of a transparent viewing panel (203), to enable an operator to position the device centrally over the subject's breast. The video capturing device (502) is in data communication with the computing device (106) via any suitable data connection (504), such as a standard USB connection, WI-FI™ connection, or the like. The video capturing device (502) captures video data and transmits the video data to the computing device (106) via the data connection (504). The computing device (106) displays the video data on the display (114) thereof. This may enable real-time video images of the breast to appear on the display (114) of the computing device (106). By eliminating the viewing panel (203), the device (500) may be made more compact.

For the imaging device to be portable it should be compact and light weight such that an operator could easily carry and operate the device for screening operations. Accordingly, the components used in the imaging device should, preferably, be light weight, compact components. The table below shows some of the suggested physical properties of components of the imaging device:

TABLE 1
Component properties

| | Weight | Length | Width | Thickness |
|---|---|---|---|---|
| Component | (grams) | (mm) | (mm) | (mm) |
| Computing device | 544 | 245 | 175 | 8.3 |
| (tablet computer) | | | | |
| Beamformer | 573 | 198 | 134 | 26 |
| Scanning element | 260 | 262 | 25 | 25 |
| (ultrasound probe) | | | | |
| Connector | 154 | 110 | 60 | 23 |
| (data connector) | | | | |
| Battery | 250 | 103 | 50 | 22 |
| (rechargeable) | | | | |
| Motor | 167 | 53 | 28 | 28 |
| (stepper motor) | | | | |

The dimensions of the scanning surface (206) are chosen to be as small as possible, consistent with the dimensions of the largest biological tissue to be measured. This ensures that all the biological tissue, for example in the subject's breast, including the axillary region, is fully imaged.

It should be appreciated that the above table merely illustrates example properties of some of the components of the device, and the components do not have to be limited to these properties.

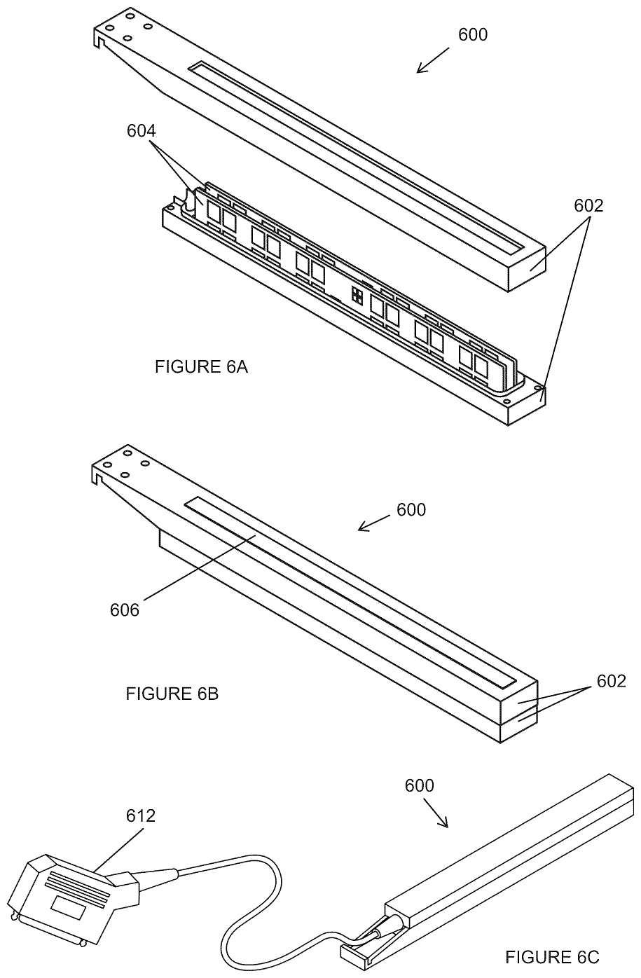

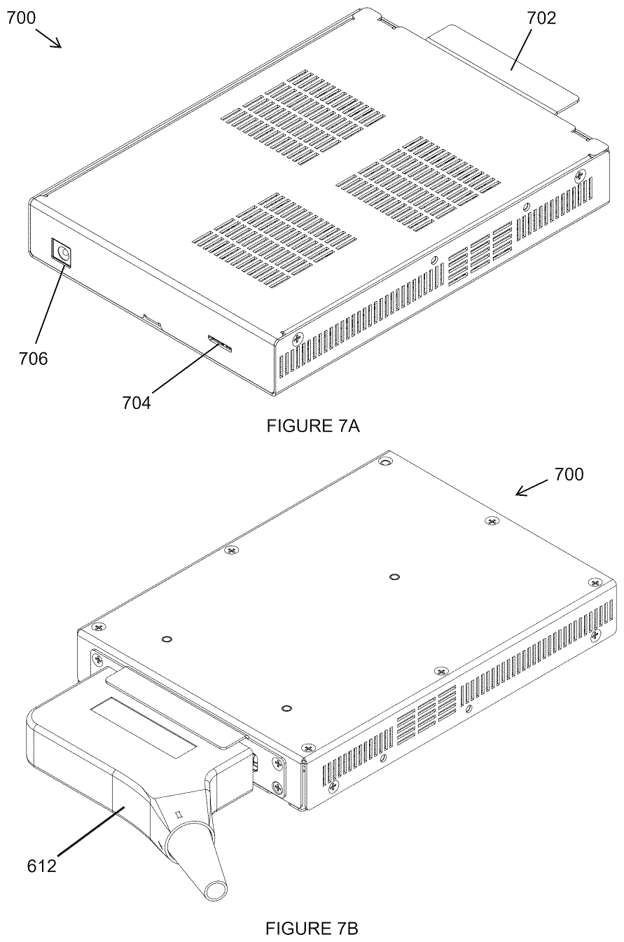

FIGS. 6A to 6C; and 7A and 7B show example embodiments of a scanning element (102) and a beamformer (700), respectively, as described in Table 1 above.

In the present embodiment the scanning element (102) is an ultrasound probe (600) comprising a probe casing (602), multiplexers (604), a piezoelectric strip (606) and a communication interface comprising a data cable terminating in a plug (612). The communication interface facilitates a wired connection and data communication between the scanning element (i.e. the ultrasound probe) and other components, such as the beamformer and/or a power supply.

The scanning element is sufficiently sized and configured to, in use, capture image data of biological tissue of varying sizes. In this embodiment, the ultrasound probe (600) has 768 elements in the piezoelectric strip (606), and there is a 12:1 multiplexer (604) that enables the 64-channel beamformer (700) to drive each element separately.

The beamformer (700) is a 64-channel beamformer in data communication with the ultrasound probe (600) by means of a transducer connector (702), such as an H-280 transducer connector. The transducer connecter (702) is configured to interface with the plug (612) of the ultrasound probe (600) and to transmit data between the ultrasound probe and the beamformer. The beamformer further includes a standard connection port (704), such as a USB-port, which facilitates connection between the beamformer and other components of the imaging device. The beamformer also includes a power input (706) which facilitates connection of the beamformer to a power source, such as a battery.

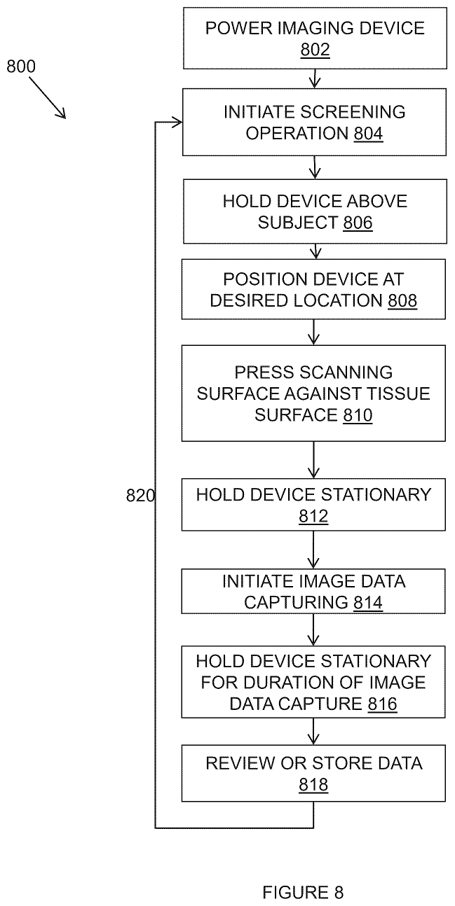

FIG. 8 is a flow diagram which illustrates an example of a method (800) of using the imaging device (200) in screening operations. The method may be conducted by an operator of the device, such as a nurse, doctor, or any other authorised person. It should be appreciated that the method (700) is merely an example method and different steps may be performed for different embodiments.

An operator may power (802) the imaging device (200) and initiate (804) a screening operation using the computing device (106). The operator may proceed to hold (806) the device (200) above a subject, such as the patient lying in a supine position, and position (808) the device into a desired location, such as the centre of the subject's nipple, using the viewing panel (203). When the operator has positioned (808) the device (200) in the desired location, the operator may gently press (810) the device (200) against a biological tissue surface, such as the surface of the patient's breast, so that the scanning surface (206) presses against the biological tissue and hold (812) the device stationary in that position. The operator then initiates (814) image data capturing by pressing the button located in the handle. The screening operation generally takes 20-30 seconds to complete, and the operator must hold (816) the device stationary in the desired position until the operation is complete.

The processed data may then be displayed to the operator via the display (114) of the computing device (106). The operator may proceed to review (818) the processed data and/or store the data to be analysed by an authorised person at a later time. Alternatively, the operator may repeat (820) the process from the initiate step (804) if necessary.

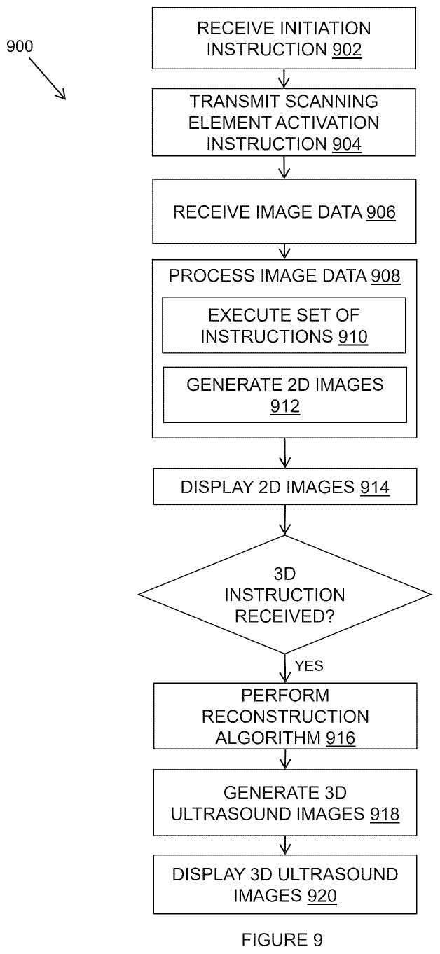

A method (900) conducted at the computing device (106) for image data capturing in a portable imaging device (100, 200, 500) is shown in the flow-diagram of FIG. 9. The flow diagram explains the steps and functions performed by the computing device (106) during the capturing of image data.

The computing device (106) may receive (902) an initiation instruction when the button on the handle is pressed by an operator of the device (200/500) and transmits (904) a scanning element activation instruction to the controller which controls the drive system (212) and the scanning element (102). The drive system (212) may then move the scanning element linearly from side-to-side across the scanning surface while the scanning element scans the biological tissue. The scanning element (102) may transmit captured image data to the electronics module (104) where the data is filtered, amplified, and converted into a readable format and transmitted, via a computing device interface, to the computing device (106) which receives (906) the image data.

The computing device (106) may process (908) the image data, which includes the computing device executing (910) a set of instructions on the image data and generating (912) a set of two-dimensional images associated with the image data captured by the scanning element (102). The set of two-dimensional ultrasound images are in the plane of the probe (102). The computing device (106) may display (914) the two-dimensional images to the operator via a display (114) of the computing device (106). In some embodiments, the computing device (106) may transmit the two-dimensional images to a storage component (108), such as a cloud storage network, where the data may be stored for subsequent use. The computing device (106) may further be configured to perform (916) a reconstruction algorithm on the captured image data and generate (918) three-dimensional ultrasound images of the biological tissue if an instruction is received from the operator of the device. The three-dimensional images may also be transmitted to the storage component (108) for subsequent use.

The computing unit (106) may display (920) the generated three-dimensional images to the operator via the user interface and display of the computing device.

It should be appreciated that in some embodiments, only one of 2D generating step (912) and the 3D generating step (918) need to be performed in a screening operation.

These three-dimensional images of the biological tissue may be sectioned to view the biological tissue in three orthogonal anatomical planes (sagittal, coronal, horizontal), and thereby aid the diagnosis of, for example, lesions. Since the data has been converted into a digital format, the data lends itself to the application of computer-assisted diagnosis (CAD), using machine learning algorithms and/or artificial intelligence, which may be performed on the computing device or remotely.

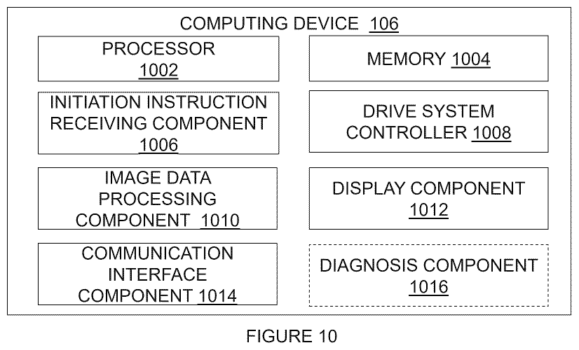

Functional components of the computing device (106) are shown in the high-level block diagram in FIG. 10. The computing device (106) includes a processor (1002) for executing the functions of components described below, which may be provided by hardware or software units executing on the computing unit. The software units are stored in a memory (1004) which provide instructions to the processor (1002) to carry out the functionality of the described components.

The computing device (106) includes an initiation instruction receiving component (1006) arranged to receive an initiation instruction indicating that a screening operation is being conducted. The initiation instruction is transmitted to the computing device (106) by a switch mechanism in response to a button being pressed by an operator.

The computing device (106) includes a drive system controller (1008) configured to control a drive system (212) for moving the scanning element from one side of the device (200/500) to the other. The drive system controller (908) is configured to send instructions to a plurality of components, such as the motor or the scanning element.

The computing device (106) includes an image data processing component (1010) configured to process image data received from the scanning element (102) and execute a set of instructions on the image data to generate a set of two-dimensional images associated with the image data captured by the scanning element. The image data processing component (1010) is further configured to execute a set of instructions, such as a reconstruction algorithm, on the processed image data to generate three-dimensional images associated with the data captured by the scanning element.

The computing device (106) includes a display component (1012) arranged to display the processed image data to the operator of the device (200/500).

The computing device (106) includes a communications interface component (1014) configured to transmit at least one of the raw image data and/or the image data in processed form to a storage component (108) accessible to the imaging device for subsequent analysis.

It should further be appreciated that in some embodiments the computing device (106) may optionally include a diagnosis component (1016) configured to perform computer-assisted diagnosis (CAD) on the processed image data using machine learning algorithms and artificial intelligence so as to assist in diagnosis of a subject. In a preferred embodiment, CAD is performed by a server computer, as described in more detail below, where the server computer may have greater processing capabilities.

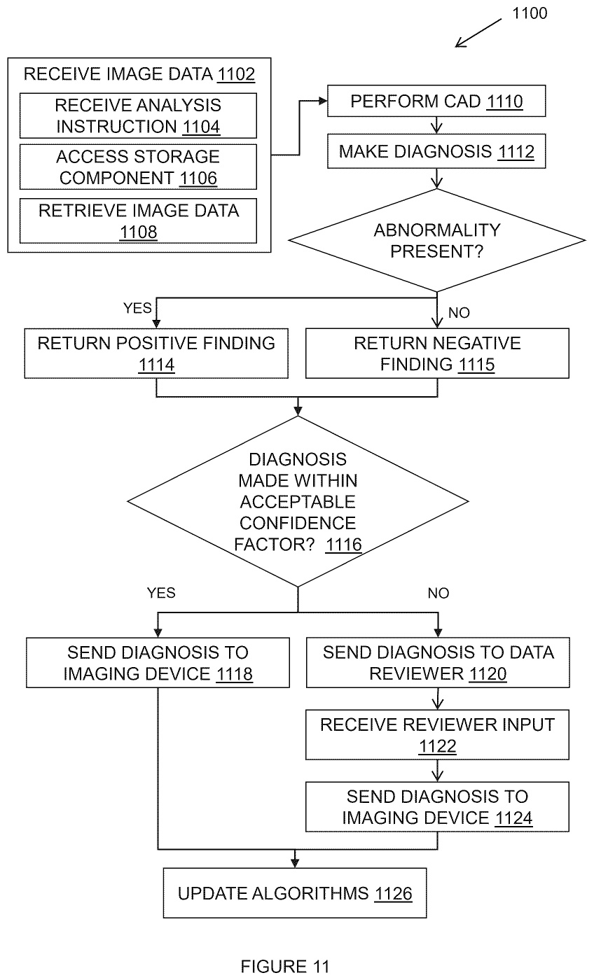

Referring to FIG. 11, a flow diagram shows an example method (1100) carried out at a server computer in a system for imaging and evaluating biological tissue.

As described with reference to FIGS. 8 and 9, an operator may use the imaging device to capture image data of biological tissue. The imaging device may transmit the image data, including at least one of raw image data and/or processed image data, to a storage component. A data reviewer may input instructions to the server computer to review image data captured by the imaging device. It should be appreciated that in some embodiments the server computer may receive an automatic notification that image data has been captured and stored in the storage component for subsequent analysis. The server component may, for example, identify the image data to be analysed by means of a data time-stamp or a unique identifier associated with a particular image device. The server computer may receive (1102) the image data of biological tissue captured by the imaging device. Receiving the image data may include the server computer receiving (1104) an analysis instruction and access (1106) the storage component to retrieve (1108) the image data captured by the imaging device (200/500). In some embodiments the server computer may receive the analysis instruction from the imaging device in response to the imaging device capturing the image data. In an alternative embodiment, the analysis instruction may be received from the imaging device in response to an analysis instruction input into the imaging device by an operator. The operator input may be transmitted to the server computer. Although not shown, in some embodiments receiving (1102) the image data may include the server computer receiving the image data from the imaging device in real-time.

Once the image data has been received (1102) the server computer may perform (1110) computer-assisted diagnosis (CAD) on the image data. This may include executing either one or both of machine-learning algorithms and artificial intelligence algorithms on the image data configured to identify if an abnormality is present in the image data. The server computer may then make (1112) a diagnosis on the CAD taking any abnormalities detected into consideration. This may include the server computer returning a positive finding (1114) if an abnormality is identified in the image data, and a negative finding (1115) if no abnormality is identified in the image data.

The server computer may determine (1116) whether the diagnosis is made within an acceptable confidence factor. For example, the server computer may be configured to assign a confidence factor to each diagnosis, and if the confidence factor of the diagnosis is above a pre-defined threshold, such as 95%, the diagnosis is made within an acceptable confidence factor, and vice versa.

If the diagnosis is made within an acceptable confidence factor, the server computer may send (1118) the diagnosis to the portable imaging device. The operator of the device may receive the diagnosis via the imaging device and advise the patient accordingly. In some embodiments the operator may relay the diagnosis to a human data reviewer to advise the patient accordingly and/or the operator may advise the patient to consult a skilled medical practitioner for further advice and, for example, treatment options. It should be appreciated that in instances where the operator of the device is not a qualified data reviewer, such as a skilled clinician, the diagnosis may optionally be sent to a human data reviewer for further advice.

As such, where a positive or negative finding is made with an acceptable degree of certainty, in other words within an acceptable confidence factor, the diagnosis may optionally be transmitted and displayed to the human data reviewer for review. For example, in the case of a positive finding, in other words where carcinoma or other abnormalities are detected, a clinician may want to review the diagnosis to advise a patient accordingly. In such an embodiment, the diagnosis may be sent to the portable imaging device and to the data reviewer.

If the diagnosis is not made within an acceptable confidence factor, the server computer may send (1120) the diagnosis to a data reviewer, such as a skilled clinician, for review. The skilled reviewer may review the diagnosis and provide an accept or correct instruction to the server computer. The server computer may receive (1122) the input from the reviewer to accept or correct the diagnosis and send (1124) the diagnosis to the imaging device. Similar to the scenario above, the operator of the imaging device may receive the diagnosis via the device and advise the patient accordingly. It should be appreciated that in some embodiments where the diagnosis is not made within an acceptable confidence factor, the diagnosis may be sent to the imaging device with a message to the operator to consult or wait for an input from a data reviewer for an accurate diagnosis.

The server computer may update (1126) either or both the machine learning algorithms and the artificial intelligence algorithms with data points from the diagnosis.

In light of the above, in instances where the artificial intelligence and computer-learning algorithms can determine a positive or negative finding with an acceptable degree of certainty, no reviewer intervention may be required. The server may then proceed to directly send its finding to the imaging device, where the operator may relay the finding to the subject.

However, in some embodiments, where the artificial intelligence and computer-learning algorithms cannot determine a positive or negative finding with an acceptable degree of certainty, the server computer may transmit and display the diagnosis to a qualified data reviewer, such as a skilled clinician. In this manner, input given by the skilled clinician may provide datapoints for assisted computer learning algorithms, thereby enhancing these algorithms.

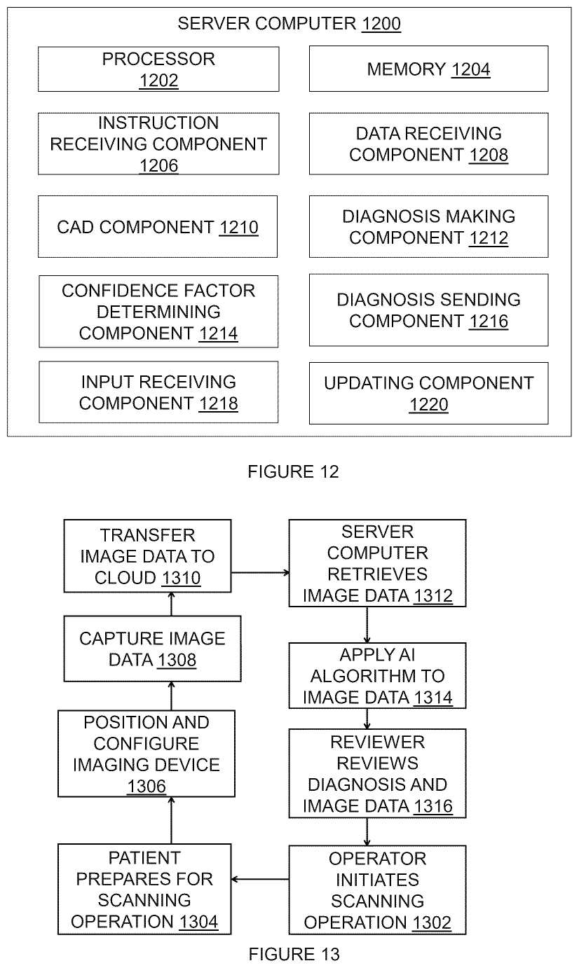

FIG. 12 is a block diagram showing example components of a server computer (1200). The server computer may include a processor (1202) for executing the functions of components described below, which may be provided by hardware or software units executing on the server computer. The software units may be stored in a memory (1204) which provide instructions to the processor (1202) to carry out the functionality of the described components.

The server computer (1200) may include an instruction receiving component (1206) for receiving an analysis instruction to access and analyse image data associated with biological tissue. In order to analyse the image data, the server computer (1200) may include a data receiving component (1208) for receiving the image data of biological tissue.

The server computer (1200) may include a CAD component (1210) for performing computer-assisted diagnosis (CAD) on the image data to identify abnormalities in the image data. Performing CAD may include executing either or both of machine learning algorithms and artificial intelligence algorithms on the image data.

The server computer (1200) may include a diagnosis making component (1212) for making a diagnosis on the CAD. The diagnosis making component (1212) may consider identified abnormalities, if any, in making a diagnosis. Making the diagnosis may include the server computer returning a positive finding if an abnormality is identified in the image data, and a negative finding if no abnormality is identified in the image data.

The server computer (1200) may include a confidence factor determining component (1214) for determining whether the diagnosis is made within an acceptable confidence factor or not. The server computer (1200) may include a diagnosis sending component (1216) for sending the diagnosis to the portable imaging device, if the diagnosis is within an acceptable confidence factor, or for sending the diagnosis to a data reviewer if the diagnosis is not within an acceptable confidence factor. The server computer (1200) may further include an input receiving component (1218) for receiving input from the reviewer to accept or correct the diagnosis. The accepted or corrected diagnosis, whichever the case may be, is then sent to the imaging device via the diagnosis sending component (1216).

The server computer (1200) may further include an updating component (1220) for updating either or both of the machine learning algorithms and the artificial intelligence algorithms with data points from the diagnosis.

FIG. 13 illustrates an example screening workflow. An operator, such as a nurse or technical assistant, initiates (1302) a screening operation to acquire raw image data of a healthy patient who has signed up for screening. The healthy patient prepares (1304) for the screening operation and lies in a supine position to expose the tissue, such as a breast, to be scanned. After the patient is in position, the operator positions and configures (1306) the imaging device and captures (1308) image data. The image device captures the raw image data, by means of the scanning element, which is then processed by a computing device of the image device. The image data, including at least one of the raw image data and the processed image data, is then transferred (1310) to a cloud network storage using a communication interface of the computing device (106). The image data is retrieved (1312) by a server computer, such as a centrally located data processing facility. Once the server computer has retrieved the image data, artificial intelligence (AI) algorithms are applied (1314) to the image data and, if there is a positive finding, in other words a potential abnormality in the biological tissue, a human diagnostic specialist reviews (1316) the diagnosis and image data via a display of the server computer, or any other computer device in communication with the server computer. As most findings will be negative, in other words include no abnormalities, this will not be an onerous task. When there is a positive finding, however, the reviewer can relay the result to the operator who may then refer the patient for follow-up assessment. It is of course imperative that follow-up is available.

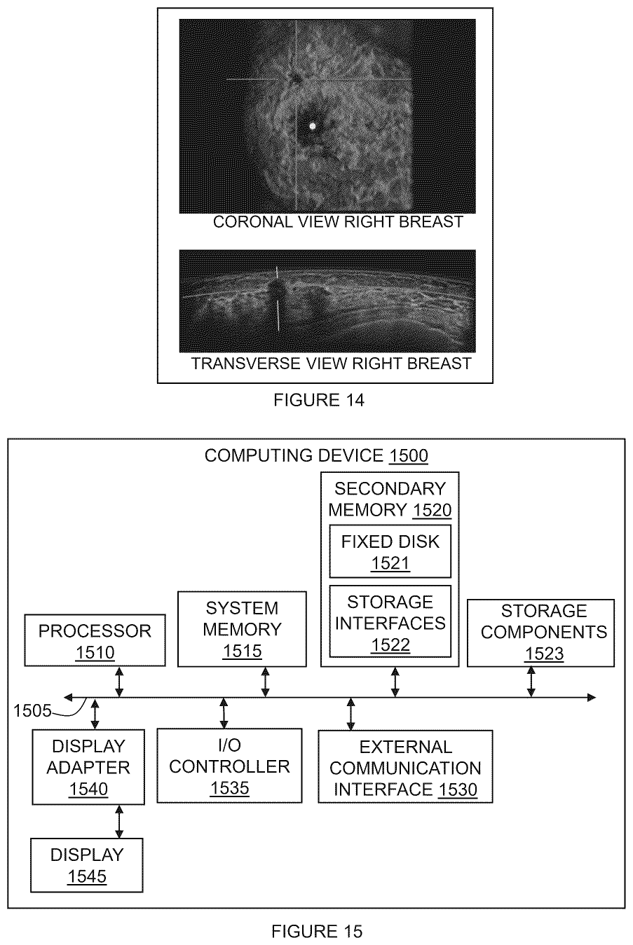

FIG. 14 illustrates example image data showing an abnormality, a malignant lesion in this case, in biological tissue in two orthogonal planes: a coronal view and a transverse view of the right breast of a subject having undergone imaging using the imaging device and methods described above.

It should be noted that the imaging devices (200, 500) are small, portable, and lightweight devices. This means that the device may be transported to rural areas, by a mobile technician, or between different clinics. This clearly promotes its accessibility and availability in the community—one of the essential factors for a successful breast-screening program enumerated above.

As at least one embodiment of the imaging device enables it to work in conjunction with a standard tablet computer or smart phone, this reduces the cost, and also long-term cost. Should the computing device (i.e. tablet or smartphone) become damaged or obsolete, it may simply be replaced with another standard tablet computer or smartphone, with the necessary configuration.

The imaging devices and methods disclosed also enable results to be rapidly available. The raw image data may be sent in near real-time (if network connectivity at a particular site allows) to a remote server for computer-aided diagnosis, with the results being rapidly returned to the imaging device for sharing with the patient.

The imaging device also requires minimal training to operate. The hand-held form-factor and the display provided by the computing device allows detailed instructions and guidance to be provided to the operator.

Being an ABUS system, the imaging devices disclosed herein also enables high sensitivity, particularly in women with dense breast tissue.

The devices and methods disclosed herein therefore promote all the essential factors for a successful breast-screening program enumerated above.

FIG. 15 illustrates an example of a computing device (1500) in which various aspects of the disclosure may be implemented, such as the computing device (106) described above, as well as the server computer (1200) described above. The computing device (1500) may be embodied as any form of data processing device including a personal computing device, a server computer, or a communication device, such as a mobile phone (e.g. cellular telephone), satellite phone, tablet computer, personal digital assistant or the like. Different embodiments of the computing device may dictate the inclusion or exclusion of various components or subsystems described below.

The computing device (1500) may be suitable for storing and executing computer program code. The various participants and elements in the previously described system diagrams may use any suitable number of subsystems or components of the computing device (1500) to facilitate the functions described herein. The computing device (1500) may include subsystems or components interconnected via a communication infrastructure (1505) (for example, a communications bus, a network, etc.). The computing device (1500) may include one or more processors (1510) and at least one memory component in the form of computer-readable media. The one or more processors (1510) may include one or more of: CPUs, graphical processing units (GPUs), microprocessors, field programmable gate arrays (FPGAs), application specific integrated circuits (ASICs) and the like. In some configurations, a number of processors may be provided and may be arranged to carry out calculations simultaneously. In some implementations various subsystems or components of the computing device (1500) may be distributed over a number of physical locations (e.g., in a distributed, cluster or cloud-based computing configuration) and appropriate software units may be arranged to manage and/or process data on behalf of remote devices.

The memory components may include system memory (1515), which may include read only memory (ROM) and random access memory (RAM). A basic input/output system (BIOS) may be stored in ROM. System software may be stored in the system memory (1515) including operating system software. The memory components may also include secondary memory (1520). The secondary memory (1520) may include a fixed disk (1521), such as a hard disk drive, and, optionally, one or more storage interfaces (1522) for interfacing with storage components (1523), such as removable storage components (e.g. magnetic tape, optical disk, flash memory drive, external hard drive, removable memory chip, etc.), network attached storage components (e.g. NAS drives), remote storage components (e.g. cloud-based storage) or the like.

The computing device (1500) may include an external communications interface (1530) for operation of the computing device (1500) in a networked environment enabling transfer of data between multiple computing devices (1500) and/or the Internet. Data transferred via the external communications interface (1530) may be in the form of signals, which may be electronic, electromagnetic, optical, radio, or other types of signal. The external communications interface (1530) may enable communication of data between the computing device (1500) and other computing devices including servers and external storage facilities. Web services may be accessible by and/or from the computing device (1500) via the communications interface (1530).

The external communications interface (1530) may be configured for connection to wireless communication channels (e.g., a cellular telephone network, wireless local area network (e.g. using Wi-Fi™), satellite-phone network, Satellite Internet Network, etc.) and may include an associated wireless transfer element, such as an antenna and associated circuitry. The external communications interface (1530) may include a subscriber identity module (SIM) in the form of an integrated circuit that stores an international mobile subscriber identity and the related key used to identify and authenticate a subscriber using the computing device (1500). One or more subscriber identity modules may be removable from or embedded in the computing device (1500).

The computer-readable media in the form of the various memory components may provide storage of computer-executable instructions, data structures, program modules, software units and other data. A computer program product may be provided by a computer-readable medium having stored computer-readable program code executable by the central processor (1510). A computer program product may be provided by a non-transient or non-transitory computer-readable medium, or may be provided via a signal or other transient or transitory means via the communications interface (1530).

Interconnection via the communication infrastructure (1505) allows the one or more processors (1510) to communicate with each subsystem or component and to control the execution of instructions from the memory components, as well as the exchange of information between subsystems or components. Peripherals (such as printers, scanners, cameras, or the like) and input/output (I/O) devices (such as a mouse, touchpad, keyboard, microphone, touch-sensitive display, input buttons, speakers and the like) may couple to or be integrally formed with the computing device (1500) either directly or via an I/O controller (1535). One or more displays (1545) (which may be touch-sensitive displays) may be coupled to or integrally formed with the computing device (1500) via a display or video adapter (1540).

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

Any of the steps, operations, components or processes described herein may be performed or implemented with one or more hardware or software units, alone or in combination with other devices. Components or devices configured or arranged to perform described functions or operations may be so arranged or configured through computer-implemented instructions which implement or carry out the described functions, algorithms, or methods. The computer-implemented instructions may be provided by hardware or software units. In one embodiment, a software unit is implemented with a computer program product comprising a non-transient or non-transitory computer-readable medium containing computer program code, which can be executed by a processor for performing any or all of the steps, operations, or processes described. Software units or functions described in this application may be implemented as computer program code using any suitable computer language such as, for example, Java™, C++, or Perl™ using, for example, conventional or object-oriented techniques. The computer program code may be stored as a series of instructions, or commands on a non-transitory computer-readable medium, such as a random access memory (RAM), a read-only memory (ROM), a magnetic medium such as a hard-drive, or an optical medium such as a CD-ROM. Any such computer-readable medium may also reside on or within a single computational apparatus, and may be present on or within different computational apparatuses within a system or network.

Flowchart illustrations and block diagrams of methods, systems, and computer program products according to embodiments are used herein. Each block of the flowchart illustrations and/or block diagrams, and combinations of blocks in the flowchart illustrations and/or block diagrams, may provide functions which may be implemented by computer readable program instructions. In some alternative implementations, the functions identified by the blocks may take place in a different order to that shown in the flowchart illustrations.

Some portions of this description describe the embodiments of the invention in terms of algorithms and symbolic representations of operations on information. These algorithmic descriptions and representations, such as accompanying flow diagrams, are commonly used by those skilled in the data processing arts to convey the substance of their work effectively to others skilled in the art. These operations, while described functionally, computationally, or logically, are understood to be implemented by computer programs or equivalent electrical circuits, microcode, or the like. The described operations may be embodied in software, firmware, hardware, or any combinations thereof.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

1. A portable imaging device for imaging biological tissue, comprising:
a scanning element including an ultrasound transducer configured to capture image data of the biological tissue of a subject;
a scanning surface configured to provide a surface serving as an interface between the scanning element and the biological tissue;
a drive system configured to automatically move the scanning element along the scanning surface, wherein the scanning element is configured to capture the image data as the scanning element is moved along the scanning surface by the drive system; and
an electronics module in data communication with the scanning element and with a computing device interface, the electronics module being configured to receive the image data of the scanning element and to transmit the image data to a computing device via the computing device interface,
wherein the computing device operatively receives the image data and presents it to an operator in a processed form.

2. The portable imaging device as claimed in claim 1, wherein the computing device is an integral computing device in data communication with the electronics module via the computing device interface.

3. The portable imaging device as claimed in claim 1, wherein the portable imaging device is configured to receive a mobile computing device, removably securable thereto, and configured for the computing device interface to be connected to the mobile computing device.

4. The portable imaging device as claimed in claim 1, wherein the computing device includes a processor for processing the image data to be presented to the operator in the processed form.

5. The portable imaging device as claimed in claim 4, wherein the image data in the processed form includes three-dimensional ultrasound images of the biological tissue which are generated by performing a reconstruction algorithm on the image data captured by the scanning element.

6. The portable imaging device as claimed in claim 1, wherein the scanning surface includes a substantially planar mesh membrane configured to be pressed against a surface of the biological tissue to compress the biological tissue and enable capture of the image data of a larger area of the biological tissue.

7. The portable imaging device as claimed in claim 1, including an optically translucent view panel, configured to provide the operator of the portable imaging device with an unobstructed view of the surface of the biological tissue, and thereby enable the operator to position the portable imaging device at a desired position with reference to the biological tissue to be scanned.

8. The portable imaging device as claimed in claim 1, including a video capturing device in data communication with the computing device which enables real-time display of the biological tissue on a display of the computing device, and thereby enables the operator to position the portable imaging device at a desired position with reference to the biological tissue being scanned.

9. The portable imaging device as claimed in claim 1, wherein the electronics module includes an analog front end configured to receive the image data as an output from the scanning element and filter, amplify and/or convert the image data from an analog to digital format.

10. The portable imaging device as claimed in claim 1, wherein the electronics module includes a digital beamformer and, wherein the digital beamformer is an ultrasound beamformer configured to transmit and receive ultrasound waves of the scanning element for improved image data quality.

11. The portable imaging device as claimed in claim 1, including a pair of handles for manually carrying or moving the portable imaging device, wherein at least one of the pair of handles has a switch mechanism incorporated therein for controlling image data capture of the scanning element.

12. The portable imaging device as claimed in claim 1, including a controller arranged to control the drive system so as to move the scanning element continuously through a range of pre-selected positions to capture the image data, wherein the image data is two-dimensional ultrasound image data.

13. The portable imaging device as claimed in claim 1, wherein the computing device includes a communications interface for operation of the computing device in a networked environment so as to enable transfer of data between the computing device and one or both of the portable imaging device and a server computer.

14. A method for imaging and evaluating biological tissue, the method being performed at a server computer and comprising:
receiving image data of the biological tissue captured by a portable imaging device;
performing computer-assisted diagnosis (CAD) on the image data byf machine learning or artificial intelligence processing to identify a presence of one or more abnormalities in the image data;
based on the one or more abnormalities in the image data, making a diagnosis on the server computer;
determining that the diagnosis is made within an acceptable confidence factor and sending the diagnosis to the portable imaging device, or
determining that the diagnosis is not made within the acceptable confidence factor and sending the diagnosis to a human data reviewer, receiving input from the human data reviewer to accept or correct the diagnosis, and sending the diagnosis to the portable imaging device; and
updating a machine learning model or an artificial intelligence model with one or more data points from the diagnosis.

15. The method as claimed in claim 14, wherein receiving the image data of the biological tissue captured by the portable imaging device further includes receiving, by the server computer, an analysis instruction, and accessing a storage component to retrieve the image data.

16. The method as claimed in claim 14, wherein making the diagnosis on the server computer further includes:
returning a positive finding if one or more abnormalities are present in the image data; or
returning a negative finding if no abnormalities are present in the image data.

17. The method as claimed in claim 14, wherein the diagnosis is made within the acceptable confidence factor if a negative finding is returned, and the diagnosis is not made within the acceptable confidence factor if a positive finding is returned.

18. The method as claimed in claim 14, wherein the diagnosis is made within the acceptable confidence factor if a confidence factor is higher than a pre-determined threshold, and the diagnosis is not made within the acceptable confidence factor if the confidence factor is lower than the pre-determined threshold.

19. A system for imaging and evaluating biological tissue, including a server computer having a memory for storing computer-readable program code and a processor for executing the computer-readable program code, the server computer comprising:
a data receiving component for retrieving image data of the biological tissue captured by a portable imaging device;
a computer-assisted diagnosis (CAD) component for performing CAD on the image data by machine learning or artificial intelligence processing to identify a presence of one or more abnormalities in the image data;
a diagnosis-making component for making a diagnosis based on the presence of one or more abnormalities in the image data;
a confidence factor determining component for determining that the diagnosis is made within an acceptable confidence factor and sending the diagnosis to the portable imaging device, or determining that the diagnosis is not made within the acceptable confidence factor and sending the diagnosis to a human data reviewer, receiving input from the human data reviewer to accept or correct the diagnosis, and sending the diagnosis to the portable imaging device; and
an updating component for updating a machine learning model or an artificial intelligence model with one or more data points from the diagnosis.

20. The system as claimed in claim 19, including an instruction-receiving component for receiving an analysis instruction and accessing a storage component to retrieve the image data.
